(19) United States
(12) Patent Application Publication
(10) Pub. No.: US 2014/0350338 A1
(43) Pub. Date: Nov. 27, 2014

(54) ENDOSCOPE AND ENDOSCOPE SYSTEM INCLUDING SAME

(71) Applicant: Panasonic Corporation, Osaka (JP)

(72) Inventors: Shogo Tanaka, Saga (JP); Haruhiko Kohno, Fukuoka (JP)

(73) Assignee: Panasonic Corporation, Osaka (JP)

(21) Appl. No.: 14/364,368

(22) PCT Filed: Dec. 12, 2012
(86) PCT No.: PCT/JP2012/007934

§ 371 Date: Jun. 11, 2014

(30) Foreign Application Priority Data
Dec. 15, 2011 (JP) 2011-274219

(51) Int. Cl. A61B 1/00; A61B 1/05

(52) U.S. Cl. CPC A61B 1/00009; A61B 1/05
USPC 600/111

(57) ABSTRACT

An endoscope includes an insertion portion to be inserted into a subject to be observed, a first imaging unit and a second imaging unit arranged side by side in a distal end portion of the insertion portion, and a control unit which generates a 3D image from captured images taken by the first imaging unit and the second imaging unit. An optical axis of the first imaging unit can be changed to enable an image capturing area of the first imaging unit to be moved along a direction in which the first imaging unit and the second imaging unit are arranged. The endoscope includes an angle operation unit to change the inclination angle of the optical axis of the first imaging unit and an angle adjustment mechanism which changes the inclination angle of the optical axis of the first imaging unit according to an operation of the angle operation unit.

### TECHNICAL FIELD

The present invention relates to an endoscope for taking an image of an interior of a subject to be observed which cannot be observed directly from outside, and an endoscope system including the endoscope, and particularly relates to an endoscope adapted to provide three-dimensional (3D) display as well as ordinary two-dimensional (2D) display and an endoscope system including the endoscope.

### BACKGROUND ART

Endoscopes are widely used in order to observe an internal organ, etc. of a person during an operation or an inspection in medical treatment. Some of such endoscopes are configured such that an imaging unit is provided at a distal end portion of an insertion portion to be inserted into a human body and an image taken by this imaging unit is displayed on a monitor. If two imaging units are provided and a 3D monitor is used to display an image in three dimensions, the efficiency of the operation or inspection can be improved because an object such as an organ can be observed three-dimensionally.

As an endoscope which makes such 3D display possible, an endoscope is known which includes an imaging unit having a wide angle of view and another imaging unit having a narrow angle of view, where the 2D display is provided based on an image taken by the imaging unit having a wide angle of view and the 3D display is provided based on images taken by the imaging unit having a wide angle of view and the imaging unit having a narrow angle of view (refer to Patent Document 1). According to this technique, the images displayed in three dimensions allow a surgical site to be observed in detail three-dimensionally while the images displayed in two dimensions allow a wide area including the surgical site and its peripheral region to be observed.

### PRIOR ART DOCUMENT(S)

### Patent Document(S)

Patent Document 1: JP H09-005643 A

### BRIEF SUMMARY OF THE INVENTION

### Task to be Accomplished by the Invention

In the prior art technique described above, a region in an image taken by the imaging unit having a wide angle of view is cut out, where the region cut out corresponds to an image capturing area of the imaging unit having a narrow angle of view, and this cutout image and a captured image taken by the imaging unit having a narrow angle of view are used to generate a 3D image, namely, two images respectively to be seen by right and left eyes when displayed stereoscopically. However, when the endoscope is moved and a distance to the object from each imaging unit is varied, the positional relationship between the image capturing areas of the two imaging units is changed, and thus, regions of the object in the two images become inconsistent with each other and generation of a proper 3D image becomes impossible.

Further, in the prior art technique described above, the captured image taken by the imaging unit having a wide angle of view is magnified by a process of interpolating pixels and thereafter a region thereof corresponding to the image capturing area of the imaging unit having a narrow angle of view is cut out such that the cut out image covers the same area as that of the captured image taken by the imaging unit having a narrow angle of view. Thus, when images are displayed in three dimensions, the actual resolutions of the two images respectively to be seen by right and left eyes are considerably different. Viewing such images for a long time causes fatigue, and thus, there is a problem that the prior art technique is not preferable for use in an operation that lasts for an extended period of time.

The present invention is made to solve such problems of the prior art, and a primary object of the present invention is to provide an endoscope and an endoscope system including the endoscope, where the endoscope is configured such that the positional relationship between the image capturing areas of two imaging units can be maintained even when the distance to the object from the image capturing area of each imaging unit is varied and that, when images are displayed in three dimensions, a significant difference in the actual resolution between the two images respectively to be seen by right and left eyes is avoided.

### Means to Accomplish the Task

An endoscope according to the present invention includes an insertion portion to be inserted into an interior of a subject to be observed, a first imaging unit and a second imaging unit arranged side by side in a distal end portion of the insertion portion and an image processing unit which generates a 3D image from captured images taken by the first imaging unit and the second imaging unit, wherein the first imaging unit is provided such that an inclination angle of an optical axis of the first imaging unit can be changed to enable an image capturing area of the first imaging unit to be moved along a direction in which the first imaging unit and the second imaging unit are arranged.

An endoscope system according to the present invention includes the aforementioned endoscope, a first display device for displaying images in two dimensions, a second display device for displaying images in three dimensions and a display control device which causes the first display device and the second display device to display images simultaneously based on 2D image data and 3D image data, respectively, which are output from the endoscope.

### Effect of the Invention

According to the present invention, by changing the inclination angle of the optical axis of the first imaging unit, the image capturing area of the first imaging unit can be moved along the direction in which the two imaging units are arranged such that, even when the distance to the object to be imaged is varied, the positional relationship between the respective image capturing areas of the two imaging units is maintained. Therefore, it is possible to obtain a proper 3D image at all times irrespective of the distance to the object.

### BRIEF DESCRIPTION OF THE DRAWINGS

FIG. 1 is an overall configuration diagram showing an endoscope system according to the first embodiment.

FIG. 2 is a cross-sectional view showing a distal end portion 12 of an insertion portion 11.

FIG. 3 is a front view showing the distal end portion 12 of the insertion portion 11.

FIG. 4 is a schematic side view showing an angle adjustment mechanism 16 for changing an inclination angle of an optical axis of a first imaging unit 13.

FIG. 5 is a block diagram schematically showing a structure of a control system controlling the angle adjustment mechanism 16.

FIG. 6 is a block diagram schematically showing a structure of an imaging control unit 26.

FIG. 7 is an explanatory diagram showing the image processing in the image control unit 26.

FIGS. 8A and 8B are a side view and a plan view, respectively, schematically showing states of image capturing areas A1, A2 of two imaging units 13, 14.

FIGS. 9A and 9B are a side view and a plan view, respectively, schematically showing the states of the image capturing areas A1, A2 when an object distance is changed.

FIG. 10 is a block diagram showing the imaging control unit 26 in an endoscope of the second embodiment.

FIGS. 11A and 11B are a schematic side view and a schematic plan view, respectively, for explaining the way an imaging position detecting unit 62 obtains a positional relationship between the image capturing areas A1 and A2.

FIG. 12 is an explanatory diagram in the form of a graph showing changes of distances XL, XR with respect to an inclination angle θ of the optical axis of the first imaging unit 13.

FIG. 13 is a perspective view showing a principal part of an endoscope according to the third embodiment.

### EMBODIMENTS FOR CARRYING OUT THE INVENTION

In the first invention made to solve the problem described above, an endoscope includes an insertion portion to be inserted into an interior of a subject to be observed, a first imaging unit and a second imaging unit arranged side by side in a distal end portion of the insertion portion and an image processing unit which generates a 3D image from captured images taken by the first imaging unit and the second imaging unit, wherein the first imaging unit is provided such that an inclination angle of an optical axis of the first imaging unit can be changed to enable an image capturing area of the first imaging unit to be moved along a direction in which the first imaging unit and the second imaging unit are arranged.

According to the first invention, by changing the inclination angle of the optical axis of the first imaging unit, the image capturing area of the first imaging unit can be moved along the direction in which the two imaging units are arranged such that, even when the distance to the object is varied, the positional relationship between the respective image capturing areas of the two imaging units is maintained. Therefore, it is possible to obtain a proper 3D image at all times irrespective of the distance to the object.

In the second invention, the endoscope further includes an angle operation unit to be operated by a user to change the inclination angle of the optical axis of the first imaging unit and an angle adjustment mechanism which changes the inclination angle of the optical axis of the first imaging unit according to an operation of the angle operation unit.

According to the second invention, during use of the endoscope, namely, while the insertion portion is inserted into the interior of the subject to be observed, the inclination angle of the optical axis of the first imaging unit may be changed in accordance with a change of the distance to the object to be imaged, and thus, usability is improved.

In the third invention, the first imaging unit includes an optical system having a wide angle of view and the second imaging unit includes an optical system having a narrow angle of view, wherein the image processing unit generates a 2D image from a first captured image taken by the first imaging unit, obtains a cutout image by cutting out a region in the first captured image corresponding to an image capturing area of the second imaging unit, and generates a 3D image from the cutout image and a second captured image taken by the second imaging unit.

According to the third invention, the first imaging unit having a wide angle of view takes an image of a wide area of the object and the image taken by the first imaging unit is used for 2D display. A 2D image displayed makes it possible to observe a wide area of the object being imaged. Particularly, by viewing such 2D images during an operation, an assistant or a trainee can observe a wide area including the surgical cite and its surrounding area in detail, whereby assistance can be provided more effectively during the operation and the training effect can be improved. On the other hand, the second imaging unit having a narrow angle of view takes an image of a narrow area of the object and the image captured by the second imaging unit and the cutout image are used to perform the 3D display. A 3D image displayed makes it possible to observe the object being imaged in detail three-dimensionally. Particularly, by viewing the 3D image during an operation, a surgeon can recognize the surgical cite three-dimensionally, whereby the efficiency of the operation can be improved.

Further, since not only the first imaging unit takes an image of a wide area of the object but also the image capturing area of the first imaging unit can be moved by changing the inclination angle of the optical axis of the first imaging unit, it is possible for a user to observe an even wider area of the object. In particular, so long as the image capturing area of the first imaging unit is moved within an extent that the image capturing area of the second imaging unit is included in the image capturing area of the first imaging unit, the 3D image does not change, and thus, during a surgical operation, the display region of the 2D image may be freely moved as desired by an assistant or a trainee without moving the display region of the 3D image which is to be viewed by a surgeon.

In the fourth invention, the first imaging unit includes an image sensor having a high resolution and the second imaging unit includes an image sensor having a low resolution.

According to the fourth invention, since the first imaging unit including an optical system having a wide angle of view includes an image sensor having a high resolution, it is possible to observe a wide area of the object in higher detail. Since the second imaging unit includes an image sensor having a low resolution and thus the second imaging unit can be made compact in size, it is possible to reduce the outer diameter of the distal end portion of the insertion portion. In addition, since the first imaging unit includes an image sensor having a high resolution and thus the first imaging unit has a large size, it is possible to easily provide the angle adjustment mechanism such that the angle adjustment mechanism does not increase the outer diameter of the distal end portion of the insertion portion.

In the fifth invention, the first imaging unit and the second imaging unit are set such that the cutout image and the second captured image have substantially the same number of pixels.

According to the fifth invention, when an image is displayed in three dimensions, actual resolutions of the two images respectively to be seen by right and left eyes become substantially the same, and thus, it is possible to reduce fatigue resulting from viewing 3D images for a longtime.

In the sixth invention, the endoscope further includes a control unit which controls the inclination angle of the optical axis of the first imaging unit such that the image capturing area of the first imaging unit and the image capturing area of the second imaging unit have a predetermined positional relationship.

According to the sixth invention, an operation to align the positions of the image capturing area of the first imaging unit and the image capturing area of the second imaging unit becomes unnecessary, and thus, usability is improved.

In the seventh invention, the control unit compares the first captured image and the second captured image to detect positional relationship between the image capturing area of the first imaging unit and the image capturing area of the second imaging unit, and controls the inclination angle of the optical axis of the first imaging unit based on a result of the detection.

According to the seventh invention, since it is possible to detect the positional relationship between the image capturing areas of the two imaging units without providing an additional sensor or the like specifically used therefor, the inclination angle of the optical axis of the first imaging unit can be properly controlled without complicating the structure.

In the eighth invention, the endoscope includes an insertion portion to be inserted into an interior of a subject to be observed, a first imaging unit and a second imaging unit arranged side by side in a distal end portion of the insertion portion and an image processing unit which generates a 3D image from captured images respectively taken by the first imaging unit and the second imaging unit, wherein the first imaging unit includes an optical system having a wide angle of view and an image sensor having a high resolution, wherein the second imaging unit includes an optical system having a narrow angle of view and an image sensor having a low resolution, wherein the image processing unit generates a 2D image from a first captured image taken by the first imaging unit, obtains a cutout image by cutting out a region in the first captured image corresponding to an image capturing area of the second imaging unit, and generates a 3D image from the cutout image and a second captured image taken by the second imaging unit, and wherein the first imaging unit and the second imaging unit are set such that the cutout image and the second captured image have substantially the same number of pixels.

According to the eighth invention, when an image is displayed in three dimensions, actual resolutions of the two images respectively to be seen by right and left eyes become substantially the same, and thus, it is possible to reduce fatigue resulting from viewing a 3D image for a long time.

In the ninth invention, an endoscope system includes the aforementioned endoscope, a first display device for displaying an image in two dimensions, a second display device for displaying images in three dimensions, and a display control device which causes the first display device and the second display device to display images simultaneously based on 2D image data and 3D image data, respectively, which are output from the endoscope.

According to the ninth invention, during an operation, by viewing the screen of the second display device showing images in three dimensions, a surgeon can recognize the surgical cite in three dimensions, and thus, the efficiency of the operation can be improved. In the meanwhile, an assistant or a trainee can view the screen of the first display device showing the images in two dimensions, and thus, assistance can be provided more effectively during the operation and the training effect can be improved.

In the following, embodiments of the present invention will be described with reference to the drawings.

### First Embodiment

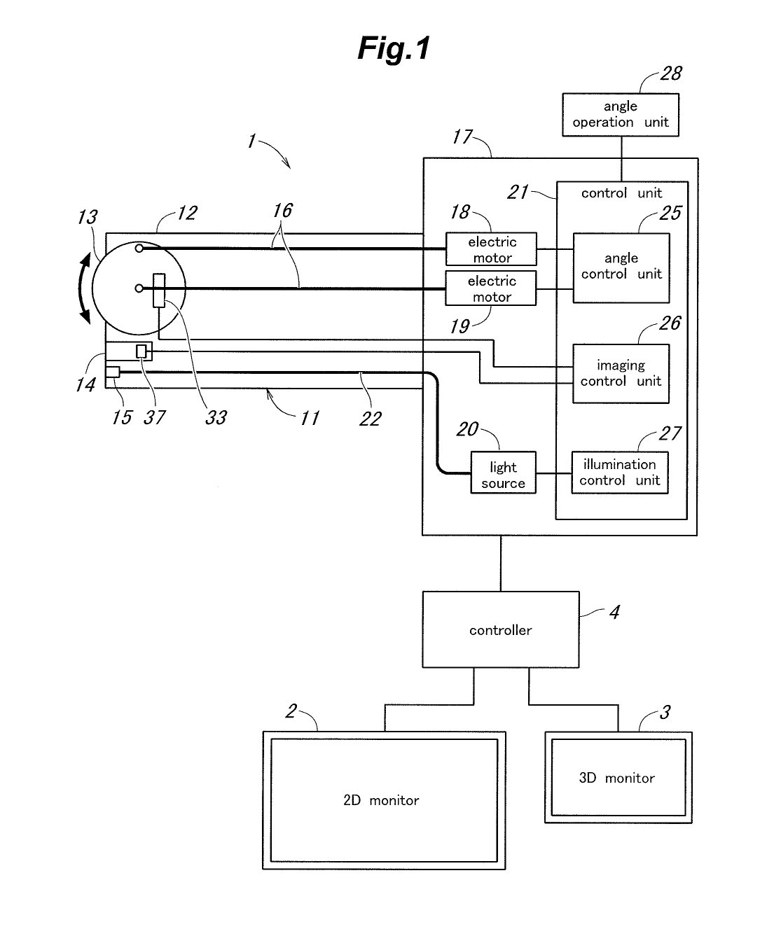

FIG. 1 is an overall configuration diagram showing an endoscope system according to the first embodiment. The endoscope system includes an endoscope 1 to be inserted into a human body (a subject to be observed) to take an image of an object such as an internal organ in the body, a 2D monitor (a first display device) 2 for displaying an image in two dimensions, a 3D monitor (a second display device) 3 for displaying an image in three dimensions and a controller (a display control device) 4 for controlling display of images on the 2D monitor 2 and the 3D monitor 3.

The endoscope 1 is what is called a rigid endoscope and its insertion portion 11 to be inserted into a body is not bendable. In a distal end portion 12 of the insertion portion 11 are provided side by side a first imaging unit 13 and a second imaging unit 14 for taking an image of the object. The first imaging unit 13 is provided such that an inclination angle of the optical axis thereof can be changed. An angle adjustment mechanism 16 is provided in the insertion portion 11 to change the inclination angle of the optical axis of the first imaging unit 13. Further, an illumination unit 15 for illuminating the object is provided to the distal end portion 12 of the insertion portion 11.

A main body portion 17 is provided on the side of the insertion portion 11 opposite to the distal end portion 12. The main body portion 17 includes two electric motors 18, 19 for driving the angle adjustment mechanism 16, a light source 20 for supplying illumination light to the illumination unit 15 and a control unit 21 for controlling the imaging units 13, 14, electric motors 18, 19 and light source 20. The light source 20 is composed of an LED or the like and is connected to the illumination unit 15 by an optical fiber cable 22. Light from the light source 20 is transmitted through the optical fiber cable 22 and emitted from the illumination unit 15.

The control unit 21 includes an angle control unit 25 which controlls the electric motors 18, 19 to change the inclination angle of the optical axis of the first imaging unit 13, an imaging control unit 26 which controls two imaging units 13, 14 and which processes captured images output from the imaging units 13, 14 and an illumination control unit 27 which controls the light source 20.

An angle operation unit 28 is connected to the control unit 21. The angle operation unit 28 is to be operated by a user to change the inclination angle of the optical axis of the first imaging unit 13. The angle operation unit 28 is composed of a position input device such as a joystick and a trackball. The inclination angle of the optical axis of the first imaging unit 13 is changed in accordance with an operation of the angle operation unit 28.

A controller 4 outputs display control data to the 2D monitor 2 and the 3D monitor 3 based on 2D image data and 3D image data output from the endoscope 1, such that a 2D image and a 3D image are simultaneously displayed on the 2D monitor 2 and 3D monitor 3, respectively, to thereby allow the object to be observed both two-dimensionally and three-dimensionally.

The 2D monitor 2 and 3D monitor 3 are each composed of, for example, a liquid crystal display. The 2D monitor 2 is configured to have a large size as the monitor 2 is to be viewed by a lot of people such as an assistant(s) and trainee(s) during an operation. The 3D monitor 3 is configured to have a small size as the monitor 3 is to be viewed by a small number of people such as a surgeon(s) during an operation. In particular, the 3D monitor 3 may be an HMD (Head Mounted Display) in view of the convenience of the surgeon.

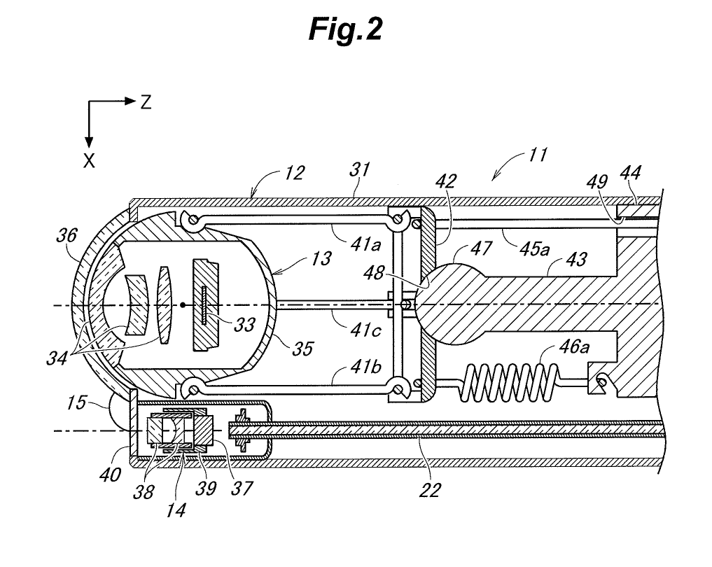

FIG. 2 is a cross-sectional view showing the distal end portion 12 of the insertion portion 11. It is to be noted that the X direction, Y direction and Z direction shown in FIG. 2 or other drawings are three directions which are perpendicular to each other.

The distal end portion 12 of the insertion portion 11 has a cylindrical cover 31 accommodating the first imaging unit 13 and second imaging unit 14 therein. The first imaging unit 13 includes an image sensor 33, an optical system 34 composed of a plurality of lenses, and a holder 35 for holding them. The holder 35 of the first imaging unit 13 is pivotally supported by the cover 31. A cover glass 36 is provided on a distal side of the first imaging unit 13. The second imaging unit 14 includes an image sensor 37, an optical system 38 composed of a plurality of lenses, and a holder 39 for holding them. A cover glass 40 is provided on a distal side of the second imaging unit 14.

The optical system 34 of the first imaging unit 13 is configured to have a wide angle of view and the optical system 38 of the second imaging unit 14 is configured to have a narrow angle of view. The first imaging unit 13 has an angle of view (Field of View) of 150 degrees and the second imaging unit 14 has an angle of view of 50 degrees, for example.

The image sensor 33 of the first imaging unit 13 is configured to have a high resolution (a large number of pixels) and the image sensor 37 of the second imaging unit 14 is configured to have a low resolution (a small number of pixels). The first imaging unit 13 has a resolution (a number of pixels) of 1920×1080 (Full HD) and the second imaging unit 14 has a resolution (a number of pixels) of 320×240 (QVGA), for example. Each image sensor 33, 37 is composed of, for example, a CMOS (Complementary Metal Oxide Semiconductor).

By configuring the image sensor 37 of the second imaging unit 14 such that it has a low resolution, the second imaging unit 14 is made compact in size, and thus, it is possible to reduce the outer diameter of the distal end portion 12 of the insertion portion 11. In addition, by configuring the image sensor 33 of the first imaging unit 13 such that it has a high resolution, the first imaging unit 13 has a large size, and thus, it is possible to easily provide the angle adjustment mechanism 16 such that the angle adjustment mechanism 16 does not increase the outer diameter of the distal end portion 12 of the insertion portion 11.

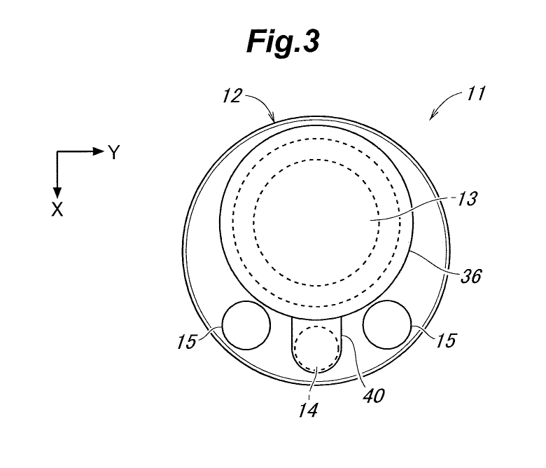

FIG. 3 is a front view showing the distal end portion 12 of the insertion portion 11. The first imaging unit 13 and the second imaging unit 14 are arranged side by side in the X direction. Two illumination units 15 are provided, one on each side of the second imaging unit 14.

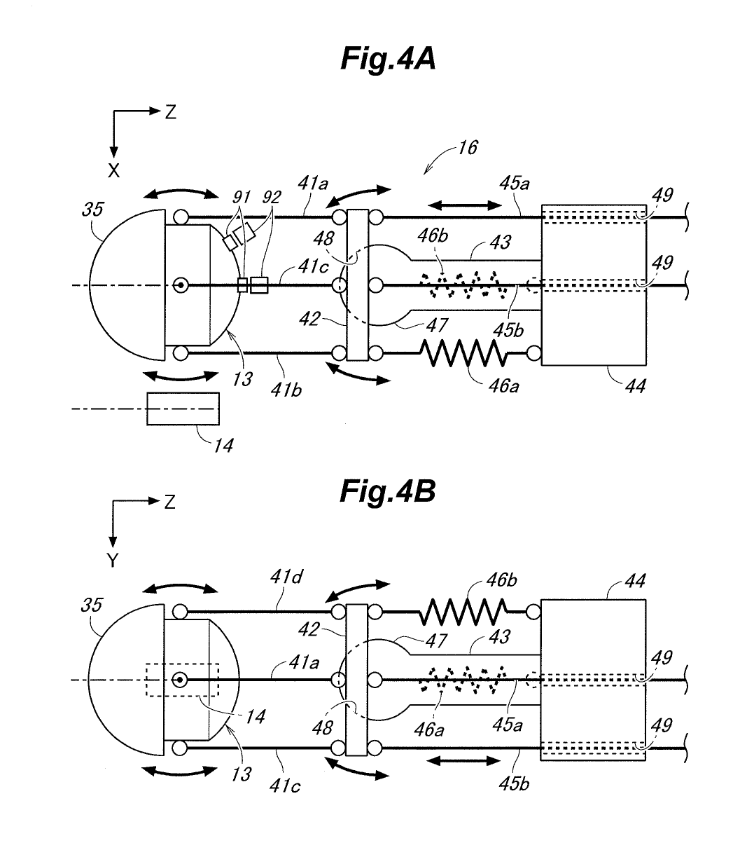

FIG. 4 is a schematic side view showing the angle adjustment mechanism 16 for changing the inclination angle of the optical axis of the first imaging unit 13. FIG. 4A and FIG. 4B show a view from the Y direction and a view from the X direction, respectively. It is to be noted that hereinafter a side of the insertion portion 11 close to the distal end portion 12 will be referred to as a front side and a side of the insertion portion 11 close to the main body portion 17 will be referred to as a rear side (refer to FIG. 1).

The adjustment mechanism 16 includes four linking rods 41a-41d connected to the holder 35 of the first imaging unit 13 at their front ends, a linking member 42 connected to rear ends of the four linking rods 41a-41d, a supporting shaft 43 which supports the linking member 42 such that the linking member 42 may be inclined around a central portion thereof, a guide member 44 which supports the supporting shaft 43, two driving rods 45a, 45b connected to the linking member 42 at their front ends, and two springs 46a, 46b which are connected to the linking member 42 at their front ends and are connected to the guide member 44 at their rear ends.

The four linking rods 41a-41d are arranged in parallel to each other so as to extend in a longitudinal direction (Z direction) of the insertion portion 11. The four linking rods 41a-41d are located circumferentially at equal intervals (90 degrees) around a central line which coincides with the optical axis of the first imaging unit 13. Two linking rods 41a, 41b are arranged in the X direction and two liking rods 41c, 41d are arranged in the Y direction.

The supporting shaft 43 includes a spherical portion 47. The central portion of the linking member 42 is provided with a receptacle 48 which has a spherical surface complementary to the spherical portion 47, whereby the linking member 42 is pivotable around a center of the spherical portion 47. The pivotal movement of the linking member 42 is transmitted to the first imaging unit 13 via the linking rods 41a-41d, and thus, the first imaging unit 13 pivots in response to the pivotal movement of the linking member 42.

Two driving rods 45a, 45b are arranged in parallel to each other so as to extend in the longitudinal direction (Z direction) of the insertion portion 11. The two driving rods 45a, 45b are located approximately on the extension of two linking rods 41a, 41c, respectively. In addition, the two driving rods 45a, 45b are inserted through through-holes 49 of the guide member 44 and are connected with the electric motors 18, 19, respectively, at the rear ends thereof (refer to FIG. 1), such that the driving rods 45a, 45b are independently driven by the respective electric motors 18, 19 so as to be advanced and retracted in the longitudinal direction.

The two springs 46a, 46b constitute pairs with two driving rods 45a, 45b, respectively. The first spring 46a and first driving rod 45a are arranged in the X direction and the second spring 46b and second driving rod 45b are arranged in the Y direction. The two springs 46a, 46b are attached to the linking member 42 and guiding member 44 in a tensioned state and urge the portions of the linking member 42 where the springs 46a, 46b are attached in the rear direction.

The urging force of the springs 46a, 46b works to pull the driving rods 45a, 45b in the forward direction while the movement of the driving rods 45a, 45b is restrained by the electric motors 18, 19, whereby the linking member 42 is kept in contact with the spherical portion 47 of the supporting shaft 43. If the electric motors 18, 19 cause the driving rods 45a, 45b to move in the backward direction against the urging force of the springs 46a, 46b, the linking member 42 pivots. If the driving rods 45a, 45b are moved in the forward direction, the linking member 42 pivots in the opposite direction.

In the angle adjustment mechanism 16 constructed as described above, if the first driving rod 45a is moved forward and backward by one of the electric motors 18, 19 as shown in FIG. 4A, the first imaging unit 13 pivots around an axis in the Y direction in response to the pivotal movement of the linking member 42, and if the second driving rod 45b is moved forward and backward by the other of the electric motors 18, 19 as shown in FIG. 4B, the first imaging unit 13 pivots around an axis in the X direction. Thus, the first imaging unit 13 can pivot around virtual two axes in the X and Y directions to change the inclination angle of the optical axis thereof in an arbitrary direction.

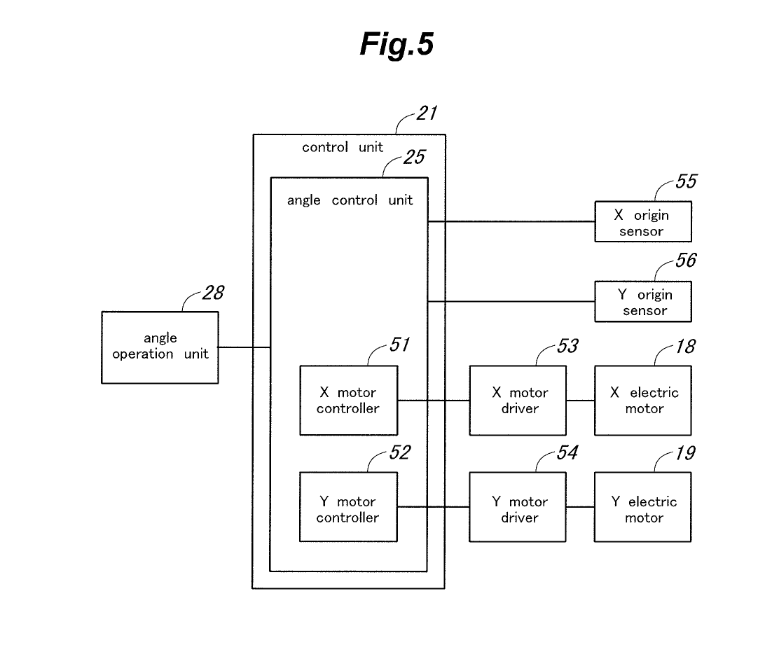

FIG. 5 is a block diagram schematically showing a structure of a control system controlling the angle adjustment mechanism 16. The angle control unit 25 of the control unit 21 includes two motor controllers 51, 52 which control the two electric motors 18, 19, respectively. The motor controllers 51, 52 output control signals to motor drivers 53, 54 to drive the electric motors 18, 19. The two electric motors 18, 19 are connected to the two driving rods 45a, 45b shown in FIG. 4, respectively, and the pivotal position of the first imaging unit 13 around each of the two axes in the X and Y directions is controlled independently.

In addition, as shown in FIG. 5, the angle control unit 25 is supplied with detection signals from two origin sensors 55, 56 and operation signals from the angle operation unit 28. The origin sensors 55, 56 detect origin positions of output shafts of the electric motors 18, 19, respectively. In the angle control unit 25, the motor controllers 51, 52 control the direction and amount of rotation of the two electric motors 18, 19 based on the detection signals from the origin sensors 55, 56 and operation signals from the angle operation unit 28.

It is to be noted that the origin position of the output shaft of each of the electric motors 18, 19 corresponds to an initial position where the optical axis of the first imaging unit 13 is parallel to the optical axis of the second imaging unit 14 which is parallel to the longitudinal direction (Z direction) of the insertion portion 11, as shown in FIG. 2. It is also to be noted that the pivotal position of the first imaging unit 13 relative to the initial position, namely, the inclination angle of the optical axis, can be controlled based on a number of driving pulses of the electric motors 18, 19 which are composed of stepping motors.

In the above-described example, the X (Y) origin sensor 55 (56) provided in the angle control unit 25 of the main body portion 17 detects the origin of pivotal movement of the imaging unit 13 and thereafter a relative rotational angle is detected based on the number of pulses applied to the electric motors 18, 19. This configuration is what is called “open loop,” in which, generally, the more complex the mechanical elements between the driving source and the object to be controlled are, the lower the effective detection accuracy is. Thus, if the detection accuracy is problematic, it is preferable to provide a magnet 91 at the bottom of the first imaging unit 13 which is pivotable and to provide a magnetic sensor 92 composed of, for example, a Hall element so as to oppose the magnet 91, as shown in FIG. 4A, such that the rotational angle is detected based on an output from the magnetic sensor 92. In this configuration, the origin is initialized based on an output from the magnetic sensor 92 when the X (Y) origin sensor 55 (56) detects the origin and the rotational angle can be obtained based on a relative change in the output from the magnetic sensor 92 thereafter. The rotational direction is uniquely determined by the control pulses output to the electric motors 18, 19. In this configuration, since a feedback loop is formed based on a detection system located very close to the object to be controlled, the rotational angle can be detected with a high accuracy and accurate positioning is possible based on the detected rotational angle.

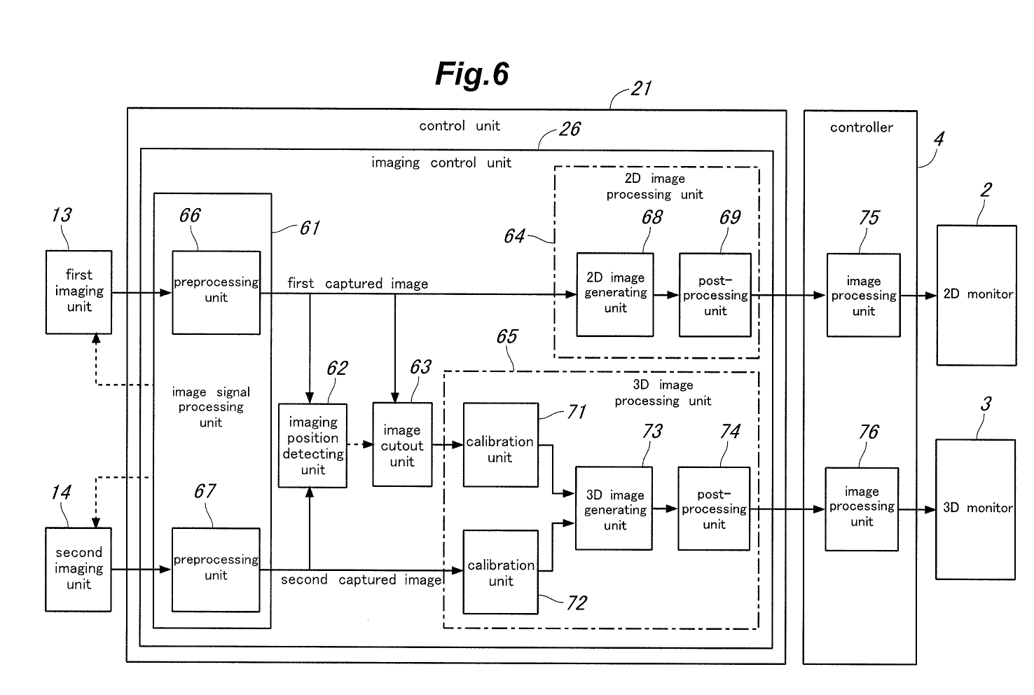

FIG. 6 is a block diagram schematically showing a structure of the imaging control unit 26. The imaging unit 26 includes an image signal processing unit 61, an imaging position detecting unit 62, an image cutout unit 63, a 2D image processing unit 64 and a 3D image processing unit 65.

The image signal processing unit 61 is composed of what is called an ISP (imaging signal processor) and includes two preprocessing units 66, 67 which perform preprocessing such as noise reduction, color correction and gamma correction. The two preprocessing units 66, 67 process the image signals output from the two imaging units 13, 14 in parallel to output a first captured image and a second captured image, respectively. In addition, the image signal processing unit 61 also has a function to operate the two imaging units 13, 14 in synchronization.

The imaging position detecting unit 62 performs a process to compare the first captured image and second captured image and to detect a positional relationship between an image capturing area of the first imaging unit 13 and an image capturing area of the second imaging unit 14. In this process, for example, feature points are extracted from each of the first captured image and second captured image, and, based on the correspondences of the feature points between the first and second captured images, a position is obtained where an image of an object of interest in the first captured image and an image of the object in the second captured image are aligned with each other.

The image cutout unit 63 performs a process to cut out a region in the first captured image of the first imaging unit 13 corresponding to the image capturing area of the second imaging unit 14 based on the positional relationship between the two image capturing areas detected by the imaging position detecting unit 62, where the first imaging unit 13 includes the optical system 34 having a wide angle of view and the image sensor 33 having a high resolution, while the second imaging unit 14 includes the optical system 38 having a narrow angle of view and the image sensor 37 having a low resolution. Thereby, the same region of the object is covered by the cutout image obtained by the image cutout unit 63 and the second captured image.

The 2D image processing unit 64 processes the first captured image to output a 2D image. The 2D image processing unit 64 includes a 2D image generating unit 68 and a post-processing unit 69. The 3D image processing unit 65 processes the second captured image and the cutout image output from the image cutout unit 63 to output a 3D image. The 3D image processing unit 65 includes two calibration units 71, 72, a 3D image generating unit 73 and a post-processing unit 74. The processes are performed in parallel in the 2D image processing unit 64 and 3D image processing unit 65 and also performed in parallel in two image processing units 75, 76 of the controller 4. Thus, a 2D image and a 3D image are simultaneously displayed on the 2D monitor 2 and 3D monitor 3, respectively.

The 3D image generating unit 65 performs a process to generate a 3D image composed of an image for the right eye and an image for the left eye. One of the cutout image and second captured image is used as the image for the right eye and the other is used as the image for the left eye.

Generally, in the technical field of stereoscopy, a calibration refers to a fixed process based on parameters for rotating images and correcting magnification errors, where the parameters have been calculated beforehand based on a result of capturing of a reference image under a specific imaging condition (condition in which a distance to an object, brightness, etc. are fixed). However, the calibration units 71, 72 perform a process to adjust the two images to be viewed by the right and left eyes such that the 3D image does not give an unnatural impression. Namely, the calibration units 71, 72 perform in real time a resizing process to match the sizes (the number of pixels in the main and sub-scanning direction) of the right and left images with each other by magnifying or reducing at least one of the images, a process of shifting at least one of the right and left images along the three-dimensional axes (X axis, Y axis and Z axis), a process of rotating at least one of the right and left images around these three axes, a process of correcting Keystone distortion which occurs in an imaging system in which optical axes of the imaging units intersect each other (crossover method), etc.

The imaging control unit 26 outputs the 2D images and 3D images as video images at a predetermined frame rate. However, it is also possible that the imaging control unit 26 outputs the 2D and 3D images as still images. In this case, super-resolution processing may be performed in which images of a plurality of frames are processed to generate a still image having a resolution higher than the original resolution.

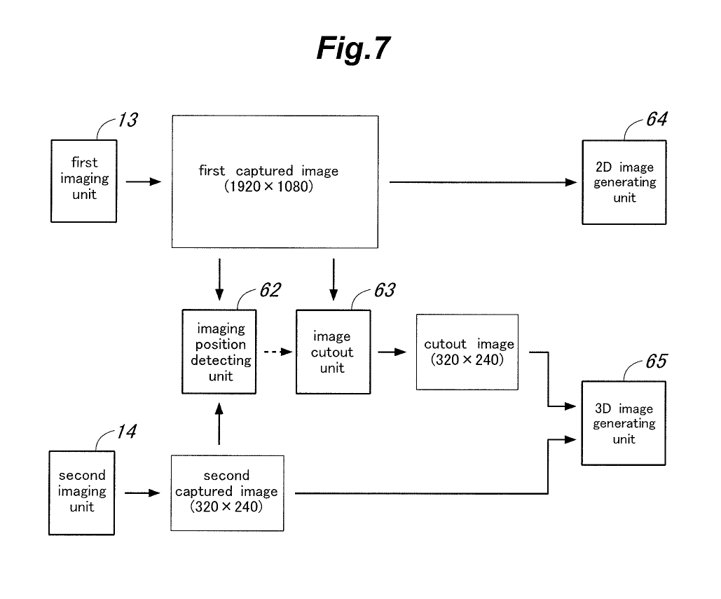

FIG. 7 is an explanatory diagram showing the image processing in the image control unit 26. In the present embodiment, the number of pixels of the image cut out from the first captured image by the image cutout unit 63 and the number of pixels of the second captured image are exactly the same (320×240 pixels, for example). Further, the positions of the image sensors 33, 37 of the two imaging units 13, 14 along the respective optical axes, the magnification rate of each optical system 34, 38, etc. (refer to FIG. 2) are adjusted such that, when the number of pixels of the cutout image and that of the second captured image are the same, the magnifications (namely, the length of an object with respect to the size in each of the main and sub-scanning directions of the screen) of the cutout image and second captured image are substantially the same.

However, the actual adjustment of the magnification, etc. is inevitably not perfect. Thus, provided that the two image sizes are set to be the same as describe above, when a region corresponding to the second captured image is cut out by the image cutout unit 63, the magnification of the resulting cutout image may be different from that of the second captured image. In this case, at least one of the images is resized by the calibration units 71, 72. In the resizing, taking into account that the both images have the same size (320×240 pixels), the magnifications are computed based on the distance between the same feature points included in each of these images, and the image with a lower magnification is magnified to be in conformity with the image with a higher magnification, where the image size is kept at the same size (320×240 pixels) by removing an unnecessary peripheral region resulting from the magnification.

It is to be noted that, due to poor adjustment of the optical systems, etc., the magnifications of the two images may be different. In such a case, if an image region corresponding to the second captured image is cut out, it is possible that the size of the resulting image is different from that of the second captured image. In this case, when an image region corresponding to the second captured image is cut out from the first captured image by the image cutout unit 63, the number of pixels of the cutout image and the number of pixels of the second captured image may be different from each other. Further, at least one of the images is resized by the calibration units 71, 72 and if the size of the cutout image is larger than 320×240 pixels, the reduction rate is computed based on the position of the same feature point(s) in the two images and the cutout image is reduced accordingly. Thereby, the image size can be kept the same and the degradation of resolution can be prevented. On the other hand, when the size of the cutout image is smaller than 320×240 pixels, the cutout image is magnified in a similar manner, whereby the image size can be kept the same.

The numbers of pixels of the first captured image and second captured image respectively depend on the resolutions of the image sensors 33, 37 which are respectively provided in the two imaging units 13, 14, and the number of pixels of the cutout image depends on the angle of view and magnification of the optical system 34 in the first imaging unit 13 and the pixel size of the image sensor 33. By properly setting these conditions, theoretically it is possible to set the number of pixels of the cutout image and the number of pixels of the second captured image to substantially the same number.

A simplified example will be described below where the pixel sizes of the image sensors 33, 37 are the same and the magnifications of the optical systems 34, 38 are the same. In a case where the first imaging unit 13 includes the image sensor 33 having the number of pixels of 1920×1080 and the second imaging unit 14 includes the image sensor 37 having the number of pixels of 320×240 as described above, the angles of view of the optical systems 34, 38 in the two imaging units 13, 14 are set such that when the first imaging unit 13 has the image capturing area of 192 mm×108 mm for a certain object, the second imaging unit 14 has the image capturing area of 32 mm×24 mm for the same object. Specifically, the positional relationship between the first imaging unit 13 and the second imaging unit 14 in the direction of the optical axes thereof and the positional relationship between the optical systems and the image sensors 33, 37 are adjusted. As a result, provided that the image sizes correspond to the respective image capturing areas, the size of a single pixel becomes 100 μm×100 μm in both the first captured image and second captured image, and thus, the actual pixel size can be the same in the cutout image and second captured image. In this case, the angle of view of the first imaging unit 13 is 140 degrees and the angle of view of the first imaging unit 13 is 50 degrees.

If the cutout image and second captured image are set to have substantially the same number of pixels as described above, when an image is displayed in three dimensions in the 3D monitor 3, the actual resolutions of the two images to be seen by right and left eyes become substantially the same. This reduces fatigue resulting from viewing a 3D image for a long time. In addition, by setting the numbers of pixels of the cutout image and second captured image to be substantially the same, the hardware resources to be used for image processing may be reduced.

It is to be noted that, as shown in FIG. 6, since the calibration units 71, 72 are provided on an output side of the image cutout unit 63 and perform an adjustment process such that the sizes of the object images in the two images are consistent with each other, the numbers of pixels of the cutout image and second captured image do not need to be exactly the same, but the numbers of pixels of the cutout image and second captured image are preferably as close to each other as possible. In addition, for a similar reason, it is not necessary for the image position detecting unit 62 to exactly determine the positional relationship between the first captured image and the second captured image and it is sufficient to determine an approximate positional relationship.

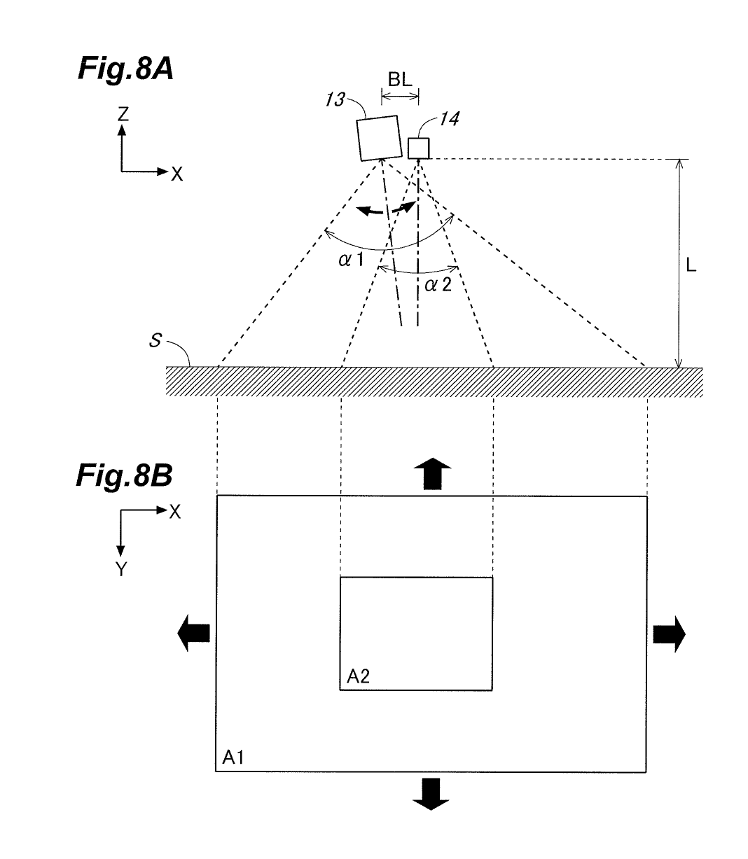

FIGS. 8A and 8B are a side view and a plan view, respectively, schematically showing states of image capturing areas A1, A2 of the two imaging units 13, 14. As described above, the first imaging unit 13 includes the optical system 34 having a wide angle of view and the second imaging unit 14 includes the optical system 38 having a narrow angle of view. Namely, the angle of view al of the first imaging unit 13 is greater than the angle of view α2 of the second imaging unit 14, and therefore, the image capturing area (hereinafter referred to as “first image capturing area” if necessary) A1 of the first imaging unit 13 is larger than the image capturing area (hereinafter referred to as “second image capturing area” if necessary) A2 of the second imaging unit 14.

The image captured by the first imaging unit 13, which captures an image of a wide area of an object S, is displayed in two dimensions and this image displayed in two dimensions makes it possible to observe a wide area of the object S. In addition, the first imaging unit 13 includes the image sensor 33 having a high resolution, and thus, makes it possible to observe a wide area of the object S with a high resolution. Therefore, in a case where an assistant or a trainee views the image during an operation, they can observe a wide area including the surgical cite and its surrounding in detail, and this can allow assistance to be provided more effectively during the operation and can improve the training effect.

On the other hand, the image captured by the second imaging unit 14, which captures an image of a narrow area of the object S, is used to display an image in three dimensions and this image displayed in three dimensions allows the object S to be observed in detail three-dimensionally. Therefore, in a case where a surgeon views the image during an operation, the surgeon can recognize the surgical cite three-dimensionally, and thus, it is possible to reduce risks and to improve the efficiency of the operation.

In addition, since the first imaging unit 13 can be pivoted around two axes which respectively extend in the X direction and Y direction, the inclination of the optical axis thereof can be changed in an arbitrary direction, and thus, the first image capturing area A1 may be moved in an arbitrary direction. In other words, if the first imaging unit 13 is pivoted around an axis extending in the X direction, the first image capturing area A1 is moved in the Y direction and if the first imaging unit 13 is pivoted around an axis extending in the Y direction, the first image capturing area A1 is moved in the X direction. If the first imaging unit 13 is pivoted around two axes respectively extending in the X direction and Y direction, the first image capturing area A1 is moved in an oblique direction.

Therefore, if a user operates the angle operation unit 28 while viewing the 2D monitor 2 to change the inclination angle of the optical axis of the first imaging unit 13, the user can move the first image capturing area A1 in a desired direction, whereby the user can observe a wider area of the object S. In particular, if the first image capturing area A1 is moved within a range where the second image capturing area A2 is included in the first image capturing area A1, the movement of the first image capturing area A1 does not change the 3D image as the second image capturing area A2 is not moved. Thus, during a surgical operation, the display region of the 2D image can be freely moved as desired by an assistant or a trainee without moving the display region of the 3D image which is to be viewed by a surgeon.

It is to be noted that, as the first image capturing area A1 moves, the image cutout unit 63 cuts out an image from a different part of the first captured image. In this case also, the region from which an image is cut out is determined based on the matching of the feature points between the two images.

It is also to be noted that, in order to move the display region of the 3D image to be viewed by the surgeon, namely, to move the second image capturing area A2, it is necessary to move the entirety of the distal end portion 12 of the insertion portion 11.

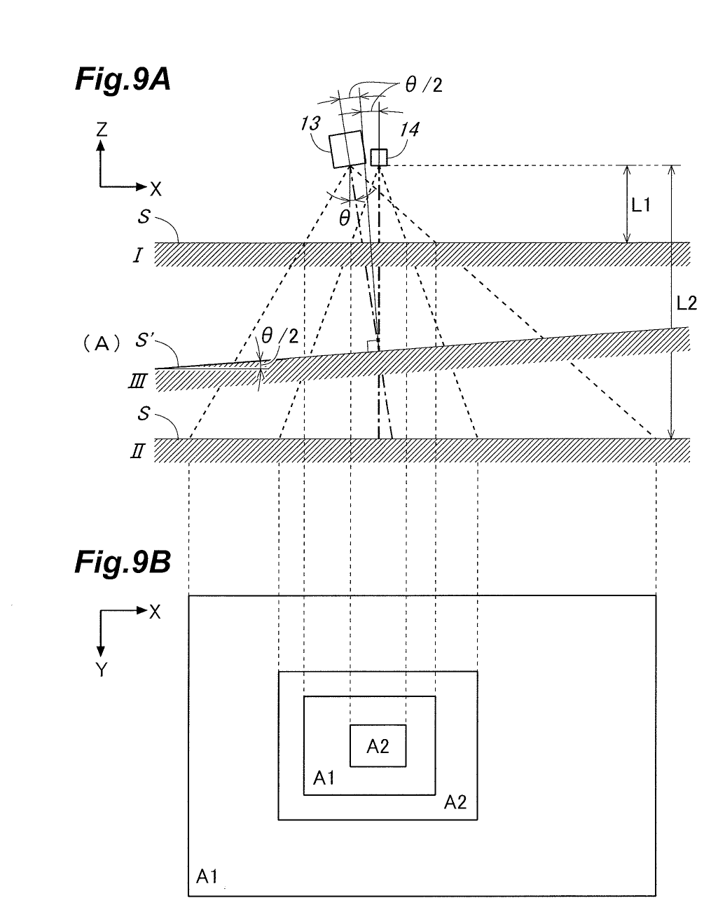

FIGS. 9A and 9B are a side view and a plan view, respectively, schematically showing the states of the image capturing areas A1, A2 when an object distance is changed. When the object distance (the distance from the imaging units 13, 14 to the object S) L is changed, the sizes of the respective image capturing areas A1, A2 of the two imaging units 13, 14 are changed and the positional relationship between the image capturing areas A1 and A2 is changed, and in particular, the first image capturing area A1 shifts in the direction in which the two imaging units 13, 14 are arranged (i.e., in the X direction as seen in FIG. 3).

In the example shown in FIG. 9, when the object S is located at a position indicated by I (object distance L1), the first image capturing area A1 is at a position biased to the left in FIG. 9 with respect to the second image capturing area A2, and when the object S is located at a position indicated by II (object distance L2), the first image capturing area A1 is at a position biased to the right in FIG. 9 with respect to the second image capturing area A2.

In this example, provided that the pivotal position of the first imaging unit 13 around the axis extending in the X direction is at the initial position, the centers of the two image capturing areas A1, A2 are at the same position with respect to the Y direction. In this state, if the first imaging unit 13 is pivoted around the axis extending in the Y direction to change the inclination angle θ of the optical axis thereof, the first image capturing area A1 is moved in the X direction, whereby the second image capturing area A2 can be located at a predetermined position in the first image capturing area A1 (e.g., at a central position).

In the example shown in FIG. 9, to locate the second image capturing area A2 at the central position in the first image capturing area A1, the inclination angle θ of the optical axis should be increased if the object S is located at the position indicated by I, while the inclination angle θ of the optical axis should be decreased if the object S is located at the position indicated by II.

Thus, by adjusting the inclination angle θ of the optical axis of the first imaging unit 13, the first image capturing area A1 can be moved in the direction in which the two imaging units 13, 14 are arranged (i.e. X direction). Therefore, even if the object distance L is varied, it is possible to keep the positional relationship between the image capturing areas A1, A2 of the two imaging units 13, 14, and thus, it is possible to always obtain a proper 3D image irrespective of the object distance L.

In the following, generation of stereoscopic images will be explained with reference to FIG. 9. To simplify the explanation, a situation is assumed where an object S′ is inclined by θ/2 with respect to a surface of another object S (horizontal surface). Under this assumption, the optical axis of each of the first imaging unit 13 and second imaging unit 14 is inclined by an angle of θ/2 with respect to the normal to the surface of the object S′. Since the optical axes of the two imaging units are respectively inclined by an equal angle with respect to the object S′, the second image capturing area A2 is present at the central position in the first image capturing area A1 on the surface of the object S′ on which the optical axes of the first imaging unit 13 and second imaging unit 14 intersect with each other. However, the point of intersection of the optical axes on this surface is projected onto the center of each of the image sensors, and thus, a parallax is zero, where a parallax is a difference in the position between pixels corresponding to a same feature point as viewed by different image sensors.

Since if the parallax is zero, the images do not provide a stereoscopic view, the 3D image processing unit 73 (refer to FIG. 6) performs a process of displacing the two images in the X direction by a predetermined number of pixels.

As described above, the rotational angle of the first imaging unit 13 is adjusted such that the second image capturing area A2 is located at the center of the first image capturing area A1, for example. The angle control unit 25 performs the control of the rotational angle by driving the electric motor 18 (refer to FIG. 1), and the rotational angle is measured by, for example, the magnetic sensor 92 explained above with reference to FIG. 4A.

Based on the measurement result of the rotational angle, the 3D image generating unit 73 (refer to FIG. 6) displaces the two images relative to each other in the X direction by an amount corresponding to a parallax that would be caused if the images were taken from locations separated from each other by a specific baseline length (e.g., an interocular distance of a human, which is supposed to be about 65 mm). Specifically, the 3D image generating unit 73 determines the amount of displacement by referring to an LUT (Lookup Table) based on the measured value of the rotational angle.

It is to be noted that if the image capturing areas A1, A2 of the two imaging units 13, 14 overlap each other at least partially, the overlapped region may be displayed in three dimensions, and thus, it is not necessarily required to locate the second image capturing area A2 at the center of the image capturing area A1 However, in order to display the entirety of the second image capturing area A2 in three dimensions, the second image capturing area A2 needs to be entirely included in the first image capturing area A1.

Since the first imaging unit 13 includes the optical system 34 having a wide angle of view to broaden the image capturing area A1, a distortion aberration tends to occur in a peripheral region of the first captured image. This distortion aberration does not cause a major problem when the image is used in displaying an image in two dimensions. However, in a case where the 3D display is performed, if a peripheral region of the first captured image including a distortion aberration is cut out and used in displaying an image in three dimensions, it may be possible that the resulting image is painful to see. Thus, when the 3D display is performed, it is preferable not to locate the second image capturing area A2 in the peripheral region of the first image capturing area A1.

### Second Embodiment

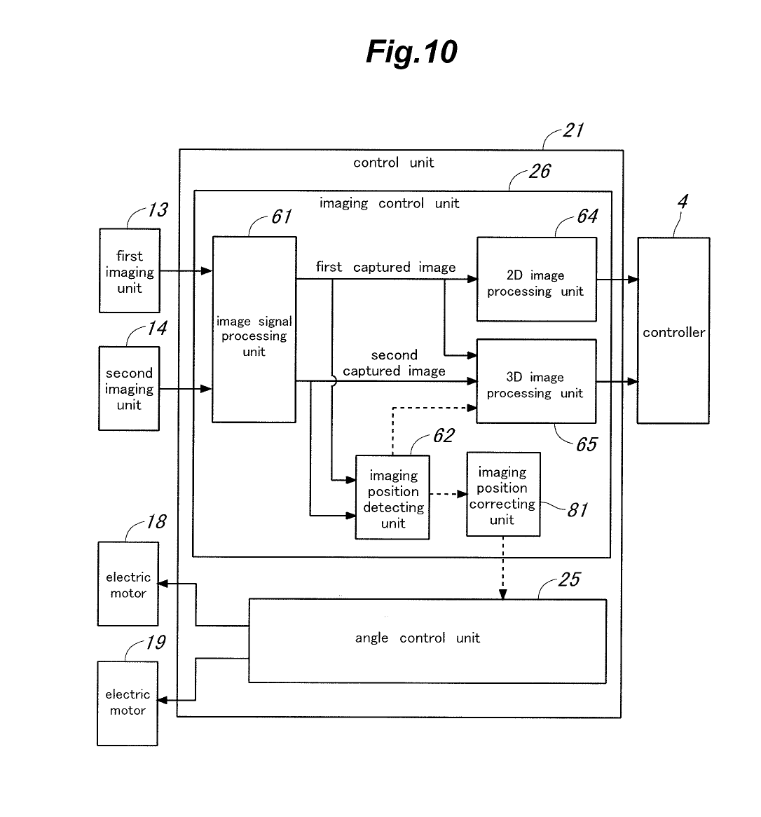

FIG. 10 is a block diagram showing the imaging control unit 26 in an endoscope according to the second embodiment. It is to be noted that the second embodiment is similar to the first embodiment except for the points noted in the following.

In this second embodiment, the control unit 21 performs control to automatically adjust the inclination angle of the optical axis of the first imaging unit 13 such that the second image capturing area is maintained at a predetermined location in the first image capturing area irrespective of the object distance. The imaging control unit 26 includes an imaging position correcting unit 81 which corrects a displacement (positional mismatch) of the image capturing area of the first imaging unit 13 with respect to the image capturing area of the second imaging unit 14, whereby an operation to adjust the position of each of the image capturing areas of the imaging units 13, 14 becomes unnecessary, and thus, usability is improved.

In a manner similar to that in the first embodiment, the imaging position detecting unit 62 compares the first captured image and the second captured image taken by the two imaging units 13, 14 to detect the positional relationship between the image capturing areas of the two imaging units 13, 14.

Based on the result of detection by the imaging position detecting unit 62, the imaging position correcting unit 81 performs a process to compute a target value of the inclination angle of the optical axis with which the displacement of the image capturing area of the first imaging unit 13 with respect to the image capturing area of the second imaging area 14 can be corrected. This target value of the inclination angle of the optical axis computed by the imaging position correcting unit 81 is output to the angle control unit 25 and the angle control unit 25 drives the electric motors 18, 19 such that the actual inclination angle of the optical axis approaches the target value. Thereby, the image capturing area of the first imaging unit 13 is moved and the image capturing area of the second imaging unit 14 is located at a predetermined position (e.g., a central position) in the image capturing area of the first imaging unit 13.

It is to be noted that in some cases it may be difficult for the imaging position correcting unit 81 to compute the inclination angle of the optical axis to correct the displacement in a single movement only by comparing the captured images, and thus, the inclination angle of the optical axis may be changed in a stepwise manner such that the change of the inclination angle of the optical axis and the comparison of the captured images are repeated alternately until the inclination angle of the optical axis is adjusted to such a value where the two image capturing areas have a predetermined positional relationship.

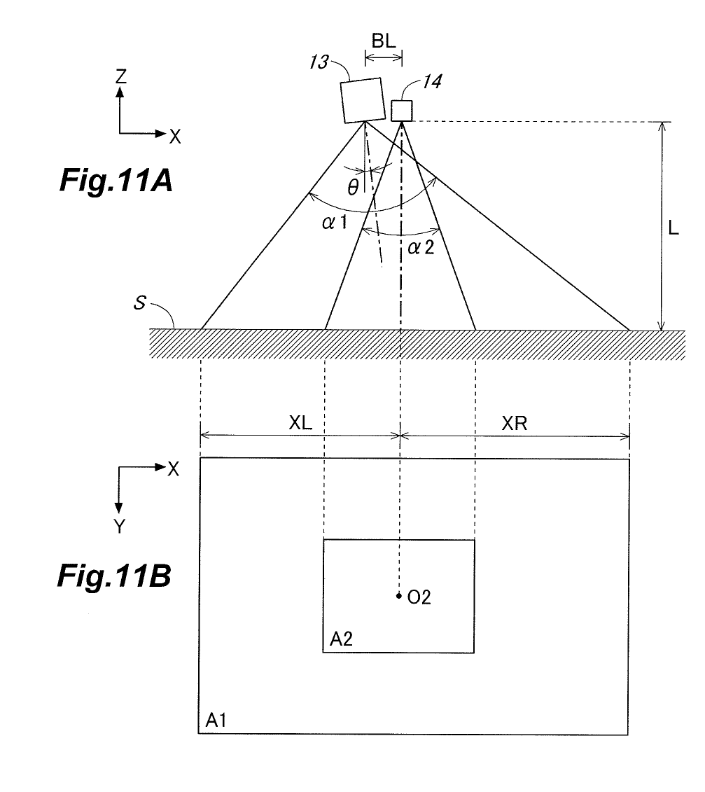

FIGS. 11A and 11B are a schematic side view and a schematic plan view, respectively, for explaining the way the imaging position detecting unit 62 obtains a positional relationship between the image capturing areas A1 and A2. It is to be noted that though the explanation below will be given in terms of the image capturing areas A1, A2, the imaging position detecting unit 62 actually performs the process based on the captured images.

Provided that the second imaging unit 14 is always directly facing to a surface of the object S to be imaged, namely, the optical axis of the second imaging unit 14 is always perpendicular to the surface of the object S to be imaged, when the object distance L is varied, the size of the second image capturing area A2 is changed, but the position of the center O2 of the second image capturing area A2 does not change. On the other hand, if the optical axis of the first imaging unit 13 is inclined, the position of the first image capturing area A1 is changed as the object distance L is varied.

In this explanation, as parameters indicating a displacement between the first and second image capturing areas A1 and A2 when position adjustment is performed to locate the second image capturing area A2 at the central part of the first image capturing area A1, a distance XL from the center O2 of the second image capturing area A2 to one of the ends (the left end in the drawing) of the first image capturing area A1 and a distance XR from the center O2 of the second image capturing area A2 to the other of the ends (the right end in the drawing) of the first image capturing area A1 will be obtained.

The distances XL, XR are defined by the following equations, where al is the angle of view of the first imaging unit 13, θ is the inclination angle of the optical axis of the first imaging unit 13 and BL is the baseline length (the distance between the two imaging units 13 and 14):

XL=L×tan(α1/2−θ)+BL (Eq. 1)

XR=L×tan(α1/2+θ)−BL (Eq. 2)

In this case, when XL and XR are substantially equal, the second image capturing area A2 is located substantially at the center of the first image capturing area A1.

Therefore, to keep the second image capturing area A2 at the center of the first image capturing area A1 irrespective of the object distance L, the distances XL, XR between the center O2 of the second image capturing A2 and the respective ends of the first image capturing area A1 are obtained. Specifically, first, the position of the second image capturing area is detected in the first image capturing area A1 by feature point matching. Subsequently, a coordinate value of the center O2 of the second image capturing area is calculated and, based on an X value of this coordinate value, XL and XR are obtained. Then, the inclination angle θ of the optical axis of the first imaging unit 13 is adjusted such that XL and XR become substantially equal.

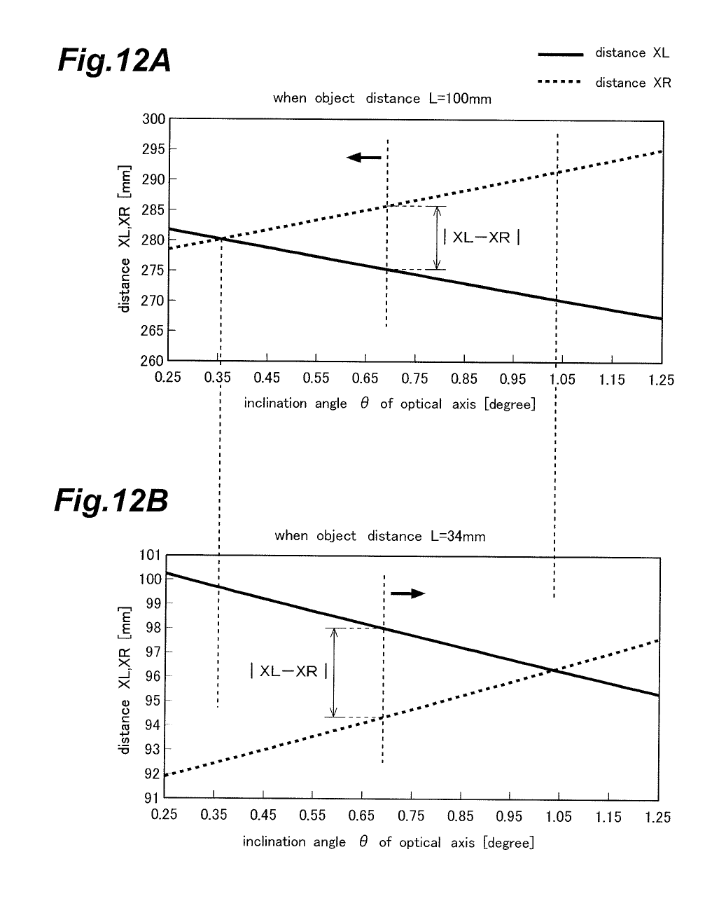

FIG. 12 is an explanatory diagram in the form of a graph showing changes of the distances XL, XR with respect to the inclination angle θ of the optical axis of the first imaging unit 13 where, FIG. 12A illustrates a case where the object distance L is 100 mm and FIG. 12B illustrates a case where the object distance L is 34 mm. It is to be noted that, in the illustrated example, the angle of view al of the first imaging unit 13 is 140 degrees and the base line length BL is 5.5 mm.

The distances XL, XR from the center O2 of the second image capturing area A2 to the respective ends of the first image capturing area A1 change depending on the inclination angle θ of the optical axis of the first imaging unit 13. As shown in FIG. 12A, in the case where the object distance L is 100 mm, when the inclination angle θ of the optical axis is set to be 0.35 degrees, the distances XL and XR are equal to each other and the second image capturing area A2 is located at the center of the first image capturing area A1. As shown in FIG. 12 (B), in the case where the object distance L is 34 mm, when the inclination angle θ of the optical axis is set to be 1.03 degrees, the distances XL and XR are equal to each other and the second image capturing area A2 is located at the center of the first image capturing area A1.

As described above, the inclination angle θ of the optical axis to locate the second image capturing area A2 at the center of the first image capturing area A1 varies for different values of the object distance L, and, to locate the second image capturing area A2 at the center of the first image capturing area A1, the inclination angle θ of the optical axis should be set such that the difference between the distances XL and XR (|XL−XR|) is decreased. Specifically, the magnitudes of the distances XL and XR obtained in the above-described manner are compared, and if XL is smaller than XR as shown in FIG. 12A, the inclination angle θ of the optical axis is decreased and if XL is larger than XR as shown in FIG. 12B, the inclination angle θ of the optical axis is increased.

It is to be noted that, in this example, the inclination angle θ of the optical axis is adjusted such that the second image capturing area A2 is located substantially at the center of the first image capturing area A1, but the positional relationship between these image capturing areas A1 and A2 is not limited thereto. Namely, it is also possible to actively maintain a state in which there is a predetermined displacement between the two image capturing areas A1 and A2. In this case, the inclination angle θ of the optical axis may be adjusted such that, for example, the ratio of the distances XL and XR (e.g., XL/XR) pertaining to the position of the first image capturing area A1 relative to the center O2 of the second image capturing area A2 is kept constant.

### Third Embodiment

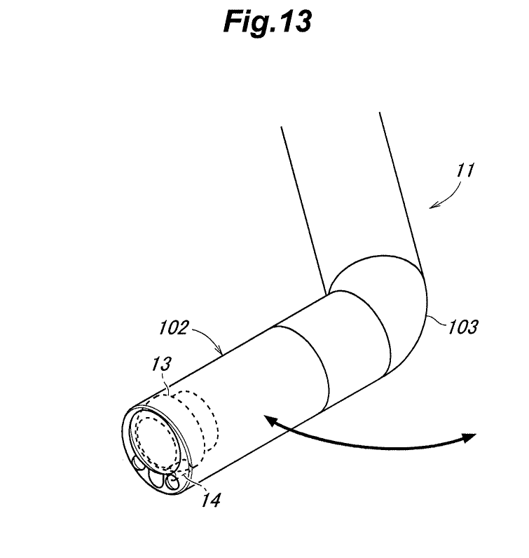

FIG. 13 is a perspective view showing a principal part of an endoscope according to the third embodiment. It is to be noted that the third embodiment is similar to the first embodiment except for the points noted in the following description.

In this third embodiment, a distal end portion 92 including a first imaging unit 13 and a second imaging unit 14 is provided to an insertion portion 91 via a bending portion 93 such that the distal end portion 92 is configured to change a direction thereof (i.e., head swinging motion). By changing the direction of the distal end portion 92 while the insertion portion 91 is inserted into an interior of the subject to be observed, it is possible to change the directions of the two imaging units 13, 14 simultaneously and to thereby observe a surgical site such as a tumor site from various directions.

In this third embodiment, similarly to the first embodiment, the endoscope may be configured to have an angle adjustment mechanism for changing the inclination angle of the optical axis of the first imaging unit 13, such that while the insertion portion 91 is inserted into an interior of the subject to be observed, the inclination angle of the optical axis of the first imaging unit 13 can be changed in addition to that the distal end portion 92 can change the direction thereof. In this case, it is necessary that the endoscope be configured such that, within a range where the distal end portion 92 may change the direction thereof, each of the bending portion 93 and angle adjustment mechanism smoothly moves. For example, the endoscope may be configured such that the first imaging unit 13 is pivoted by a flexible cable which is pushed and pulled by an electric motor.

It is to be noted that, in the foregoing embodiments, the first imaging unit 13 is configured to be pivoted around two axes to allow the inclination angle of the optical axis of the first imaging unit 13 to be changed in an arbitrary direction, but the first imaging unit 13 may be configured to be pivoted around only one axis. In this case, the first imaging unit 13 may be configured such that the image capturing area of the first imaging unit 13 can be moved in the direction in which the two imaging units 13, 14 are arranged, and in the example shown in FIG. 4, the first imaging unit 13 may be configured such that it can be pivoted around an axis in the direction (Y direction) substantially perpendicular to both the direction in which the two imaging units 13, 14 are arranged (X direction) and the direction of the optical axis of the second imaging unit 14 (Z direction). In this way, even if the object distance is varied, the positional relationship between the image capturing areas of the two imaging units 13, 14 can be kept unchanged.

In the foregoing embodiments, the first imaging unit 13 which is provided such that the inclination angle of the optical axis thereof can be changed includes the optical system 34 having a wide angle of view and the image sensor 33 having a high resolution and the second imaging unit 14 which is provided such that the inclination angle of the optical axis thereof cannot be changed includes the optical system 38 having a narrow angle of view and the image sensor 37 having a low resolution, but the present invention is not limited to such a combination. For example, the imaging unit which is provided such that the inclination angle of the optical axis thereof can be changed may include an optical system having a narrow angle of view and an image sensor having a low resolution and the imaging unit which is provided such that the inclination angle of the optical axis thereof cannot be changed may include an optical system having a wide angle of view and an image sensor having a high resolution. In this case, it is possible to move the display region of the 3D image in the fixed display region of the 2D image. However, an imaging unit including an image sensor having a high resolution is relatively large and it is easy to mount a driving mechanism for driving the imaging unit, and thus, it is preferable to provide an angle adjustment mechanism only to the imaging unit having a high resolution. This allows the angle adjustment mechanism to be mounted easily without increasing the outer diameter of the insertion portion.

In the foregoing embodiments, the angle adjustment mechanism 16 is configured to be driven by the electric motors 18, 19, but the angle adjustment mechanism 16 may be configured to be driven manually. Also, in the foregoing embodiments, the first imaging unit 13 is configured such that the inclination angle of the optical axis of the first imaging unit 13 can be changed during use, namely, while the insertion portion 11 is inserted into an interior of the subject to be observed, but the first imaging unit 13 may be configured such that the inclination angle of the optical axis thereof can be adjusted only when the endoscope is not used or the insertion portion 11 is not inserted into the interior of the subject to be observed, thereby to simplify the structure of the endoscope. In this case, the shape, size, etc. of a lesion, which is an object to be imaged, are obtained in advance using X-ray or ultrasonic waves, and based on the distance to the object, which is assumed from an operative procedure to be adopted, the angle is adjusted in advance before use or during regular maintenance.

In the foregoing embodiments, the control unit 21 provided in the main body portion 17 of the endoscope 1 performs an image processing to generate and output the 2D and 3D images from the captured images taken by the two imaging units 13, 14. However, this image processing may be performed by an image processing device separate from the endoscope 1.

In the foregoing embodiments, the endoscope is configured such that the inclination angle of the optical axis of the first imaging unit 13 can be changed so as to be able to maintain the positional relationship between the image capturing areas of the two imaging units 13, 14 even if the distance to the object is varied. However, in order to achieve only the purpose of avoiding a major difference in the actual resolutions of two images respectively to be seen by right and left eyes when the image is displayed in three dimensions, it is not necessarily required that the endoscope be configured such that the inclination angle of the optical axis of the imaging unit can be changed, and the endoscope may be configured such that the inclination angle of the optical axis of neither of the two imaging units can be changed.

Further, in the foregoing embodiments, irrespective of the object distance, the positional relationship between the image capturing areas of the two imaging units, which is necessary to perform the angle adjustment for maintaining the positional relationship between the image capturing areas of the two imaging units, is obtained by an image processing in which the two captured images are compared with each other. However, the object distance may be detected by a sensor instead of or in addition to such an image processing. For example, if the movement of the endoscope 1 is detected by an acceleration sensor, changes in the object distance can be estimated, and this allows the direction and magnitude in the changes of the inclination angle of the optical axis to be obtained so that they can be used in adjusting the angle.

### INDUSTRIAL APPLICABILITY

The endoscope and the endoscope system including the same according to the present invention have advantages that, even when the distance to an object to be imaged is varied, the positional relationship between the image capturing areas of the two imaging units can be maintained and that, when images are displayed in three dimensions, a significant difference in the actual resolution between the two images respectively to be seen by right and left eyes is avoided, and thus, are useful as an endoscope for taking an image of an interior of a subject to be observed which cannot be observed directly from outside and an endoscope system including the endoscope.

### GLOSSARY

1 endoscope
2 2D monitor (first display device)
3 3D monitor (second display device)
4 controller (display control device)
11 insertion portion
12 distal end portion
13 first imaging unit
14 second imaging unit
16 angle adjustment mechanism
21 control unit
28 angle operation unit
33, 37 image sensor
34, 38 optical system
62 imaging position detecting unit
63 image cutout unit
64 2D image processing unit
65 3D image processing unit
81 imaging position correcting unit
A1, A2 image capturing area
S object
α1, α2 angle of view
S inclination angle of an optical axis

1-9. (canceled)

10. An endoscope, comprising:
an insertion portion to be inserted into an interior of a subject to be observed;
a first imaging unit and a second imaging unit arranged side by side in a distal end portion of the insertion portion, wherein the first imaging unit includes
an optical system having a wide angle of view and the second imaging unit includes an optical system having a narrow angle of view; and
an image processing unit which generates a 3D image from captured images taken by the first imaging unit and the second imaging unit;
wherein the first imaging unit is provided such that an inclination angle of an optical axis of the first imaging unit can be changed to enable an image capturing area of the first imaging unit to be moved.

11. The endoscope according to claim 10, further comprising:
an angle operation unit to be operated by a user to change the inclination angle of the optical axis of the first imaging unit; and
an angle adjustment mechanism which changes the inclination angle of the optical axis of the first imaging unit according to an operation of the angle operation unit.

12. The endoscope according to claim 10,
wherein the image processing unit generates a 2D image from a first captured image taken by the first imaging unit, obtains a cutout image by cutting out a region in the first captured image corresponding to an image capturing area of the second imaging unit, and generates a 3D image from the cutout image and a second captured image taken by the second imaging unit.

13. The endoscope according to claim 12,
wherein the first imaging unit includes an image sensor having a high resolution; and
wherein the second imaging unit includes an image sensor having a low resolution.

14. The endoscope according to claim 13,
wherein the first imaging unit and the second imaging unit are set such that the cutout image and the second captured image have substantially the same number of pixels.

15. The endoscope according to claim 10, further comprising:
a control unit which controls the inclination angle of the optical axis of the first imaging unit such that an image capturing area of the first imaging unit and an image capturing area of the second imaging unit have a predetermined positional relationship.

16. The endoscope according to claim 15,
wherein the control unit compares the first captured image and the second captured image to detect positional relationship between the image capturing area of the first imaging unit and the image capturing area of the second imaging unit, and controls the inclination angle of the optical axis of the first imaging unit based on a result of the detection.

17. An endoscope, comprising:
an insertion portion to be inserted into an interior of a subject to be observed;
a first imaging unit and a second imaging unit arranged side by side in a distal end portion of the insertion portion; and
an image processing unit which generates a 3D image from captured images taken by the first imaging unit and the second imaging unit;
wherein the first imaging unit includes an optical system having a wide angle of view and an image sensor having a high resolution;
wherein the second imaging unit includes an optical system having a narrow angle of view and an image sensor having a low resolution;
wherein the image processing unit generates a 2D image from a first captured image taken by the first imaging unit, obtains a cutout image by cutting out a region in the first captured image corresponding to an image capturing area of the second imaging unit, and generates a 3D image from the cutout image and a second captured image taken by the second imaging unit; and
wherein the first imaging unit and the second imaging unit are set such that the cutout image and the second captured image have substantially the same number of pixels.

18. An endoscope system, comprising:
the endoscope according to claim 10;
a first display device for displaying images in two dimensions;
a second display device for displaying images in three dimensions; and
a display control device which causes the first display device and the second display device to display images simultaneously based on 2D image data and 3D image data, respectively, which are output from the endoscope.
